(19) United States
(12) Patent Application Publication
(10) Pub. No.: US 2013/0157369 A1
(43) Pub. Date: Jun. 20, 2013

(54) METHOD FOR IMPROVED TRANSFORMATION USING AGROBACTERIUM

(71) Applicant: DOW AGROSCIENCES LLC, Indianapolis, IN (US)

(72) Inventor: Paul David Miller, Salem, OR (US)

(73) Assignee: DOW AGROSCIENCES LLC, Indianapolis, IN (US)

(21) Appl. No.: 13/715,118

(22) Filed: Dec. 14, 2012

Related U.S. Application Data
(60) Provisional application No. 61/576,138, filed on Dec. 15, 2011.

(51) Int. Cl. C12N 15/82

(52) U.S. Cl. CPC C12N 15/8205
USPC 435/469

(57) ABSTRACT

Methods to increase transformation frequency in plants when using Agrobacterium as the transformant are described. The methods include exposing plant cells to Agrobacterium cells in a liquid medium containing a surfactant. Some methods include exposing the plant cells to continuous light after exposure to the Agrobacterium cells. Examples of plants useful with these methods include maize plants (e.g., immature maize embryos).

### CROSS REFERENCE TO RELATED APPLICATIONS

This application claims the benefit of U.S. Provisional Patent Application Ser. No. 61/576,138 filed Dec. 15, 2011.

### BACKGROUND

Plant transformation generally encompasses the methodologies required and utilized for the introduction of a plant-expressible foreign gene into plant cells, such that fertile progeny plants may be obtained which stably maintain and express the foreign gene. Numerous members of the monocotyledonous and dicotyledonous classifications have been transformed. Transgenic agronomic crops, as well as fruits and vegetables, are of commercial interest. Such crops include but are not limited to maize, rice, soybeans, canola, sunflower, alfalfa, sorghum, wheat, cotton, peanuts, tomatoes, potatoes, and the like.

Several techniques are known for introducing foreign genetic material into plant cells, and for obtaining plants that stably maintain and express the introduced gene. Such techniques include acceleration of genetic material coated onto microparticles directly into cells (e.g., U.S. Pat. No. 4,945,050 and U.S. Pat. No. 5,141,131). Other transformation technology includes WHISKERS™ technology (see, e.g., U.S. Pat. No. 5,302,523 and U.S. Pat. No. 5,464,765). Electroporation technology has also been used to transform plants. See, e.g., WO 87/06614, U.S. Pat. No. 5,472,869, U.S. Pat. No. 5,384,253, WO 92/09696, and WO 93/21335. Additionally, fusion of plant protoplasts with liposomes containing the DNA to be delivered, direct injection of the DNA, as well as other possible methods, may be employed.

Once the inserted DNA has been integrated into the plant genome, it is usually relatively stable throughout subsequent generations. The transformed cells grow inside the plants in the usual manner. They can form germ cells and transmit the transformed trait(s) to progeny plants. Such plants can be grown in the normal manner and may be crossed with plants that have the same transformed hereditary factors or other hereditary factors. The resulting hybrid individuals have the corresponding phenotypic properties, for example, the ability to control the feeding of plant pest insects.

A number of alternative techniques can also be used for inserting DNA into a host plant cell. Those techniques include, but are not limited to, transformation with T-DNA delivered by Agrobacterium tumefaciens or Agrobacterium rhizogenes as the transformation agent. Plants may be transformed using Agrobacterium technology, as described, for example, in U.S. Pat. No. 5,177,010, U.S. Pat. No. 5,104,310, European Patent Application No. 0131624B1, European Patent Application No. 120516, European Patent Application No. 159418B1, European Patent Application No. 176112, U.S. Pat. No. 5,149,645, U.S. Pat. No. 5,469,976, U.S. Pat. No. 5,464,763, U.S. Pat. No. 4,940,838, U.S. Pat. No. 4,693,976, European Patent Application No. 116718, European Patent Application No. 290799, European Patent Application No. 320500, European Patent Application No. 604662, European Patent Application No. 627752, European Patent Application No. 0267159, European Patent Application No. 0292435, U.S. Pat. No. 5,231,019, U.S. Pat. No. 5,463,174, U.S. Pat. No. 4,762,785, U.S. Pat. No. 5,004,863, and U.S. Pat. No. 5,159,135. The use of T-DNA-containing vectors for the transformation of plant cells has been intensively researched and sufficiently described in European Patent Application 120516; An et al., (1985, EMBO J. 4:277-284); Fraley et al., (1986, Crit. Rev. Plant Sci. 4:1-46), and Lee and Gelvin (2008, Plant Physiol. 146:325-332), and is well established in the field.

A critical first step in the transformation of plant cells by Agrobacterium spp. is close contact, binding, or adherence of the bacterial cells to the cells of the host plant to be transformed. After cell-cell binding, the biology of T-DNA transfer from Agrobacterium to plant cells is known. See, e.g., Gelvin, 2003, Microbiol. Molec. Biol. Rev. 67:16-37; and Gelvin, 2009, Plant Physiol. 150:1665-1676. At minimum, at least a T-DNA right border repeat, but often both the right border repeat and the left border repeat of the Ti or Ri plasmid will be joined as the flanking region of the genes desired to be inserted into the plant cell. The left and right T-DNA border repeats are crucial cis-acting sequences required for T-DNA transfer. Various trans-acting components are encoded within the total Agrobacterium genome. Primary amongst these are the proteins encoded by the vir genes, which are normally found as a series of operons on the Ti or Ri plasmids. Various Ti and Ri plasmids differ somewhat in the complement of vir genes, with, for example, virF not always being present. Proteins encoded by vir genes perform many different functions, including recognition and signaling of plant cell/bacteria interaction, induction of vir gene transcription, formation of a Type IV secretion channel, recognition of T-DNA border repeats, formation of T-strands, transfer of T-strands to the plant cell, import of the T-strands into the plant cell nucleus, and integration of T-strands into the plant nuclear chromosome, to name but a few. See, e.g., Tzfira and Citovsky, 2006, Curr. Opin. Biotechnol. 17:147-154.

If Agrobacterium strains are used for transformation, the DNA to be inserted into the plant cell can be cloned into special plasmids, for example, either into an intermediate (shuttle) vector or into a binary vector. Intermediate vectors are not capable of independent replication in Agrobacterium cells, but can be manipulated and replicated in common Escherichia coli molecular cloning strains. It is common that such intermediate vectors comprise sequences, framed by the right and left T-DNA border repeat regions, that may include a selectable marker gene functional for the selection of transformed plant cells, a cloning linker, cloning polylinker, or other sequence which can function as an introduction site for genes destined for plant cell transformation. Cloning and manipulation of genes desired to be transferred to plants can thus be easily performed by standard methodologies in E. coli, using the shuttle vector as a cloning vector. The finally manipulated shuttle vector can subsequently be introduced into Agrobacterium plant transformation strains for further work. The intermediate vector can be transferred into Agrobacterium by means of a helper plasmid (via bacterial conjugation), by electroporation, by chemically mediated direct DNA transformation, or by other known methodologies. Shuttle vectors can be integrated into the Ti or Ri plasmid or derivatives thereof by homologous recombination owing to sequences that are homologous between the Ti or Ri plasmid, or derivatives thereof, and the intermediate plasmid. This homologous recombination (i.e. plasmid integration) event thereby provides a means of stably maintaining the altered shuttle vector in Agrobacterium, with an origin of replication and other plasmid maintenance functions provided by the Ti or Ri plasmid portion of the co-integrant plasmid. The Ti or Ri plasmid also comprises the vir regions comprising vir genes necessary for the transfer of the T-DNA. It is common that the plasmid carrying the vir region is a mutated Ti or Ri plasmid (helper plasmid) from which the T-DNA region, including the right and left T-DNA border repeats, have been deleted. Such pTi-derived plasmids, having functional vir genes and lacking all or substantially all of the T-region and associated elements are descriptively referred to herein as helper plasmids.

The superbinary system is a specialized example of the shuttle vector/homologous recombination system (reviewed by Komari et al., 2006, In: Methods in Molecular Biology (K. Wang, ed.) No. 343: Agrobacterium Protocols, pp. 15-41; and Komori et al., 2007, Plant Physiol. 145:1155-1160). Strain LBA4404(pSB1) harbors two independently-replicating plasmids, pAL4404 and pSB1. pAL4404 is a Ti-plasmid-derived helper plasmid which contains an intact set of vir genes (from Ti plasmid pTiACH5), but which has no T-DNA region (and thus no T-DNA left and right border repeat sequences). Plasmid pSB1 supplies an additional partial set of vir genes derived from pTiBo542; this partial vir gene set includes the virB operon and the virC operon, as well as genes virG and virD1. One example of a shuttle vector used in the superbinary system is pSB11, which contains a cloning polylinker that serves as an introduction site for genes destined for plant cell transformation, flanked by Right and Left T-DNA border repeat regions. Shuttle vector pSB11 is not capable of independent replication in Agrobacterium, but is stably maintained as a co-integrant plasmid when integrated into pSB1 by means of homologous recombination between common sequences present on pSB1 and pSB11. Thus, the fully modified T-DNA region introduced into LBA4404(pSB1) on a modified pSB11 vector is productively acted upon and transferred into plant cells by Vir proteins derived from two different Agrobacterium Ti plasmid sources (pTiACH5 and pTiBo542). The Agrobacterium tumefaciens host strain employed with the superbinary system is LBA4404(pSB1). The superbinary system has proven to be particularly useful in transformation of monocot plant species. See Hiei et al., (1994) Plant J. 6:271-282; and Ishida et al., (1996) Nat. Biotechnol. 14:745-750.

In addition to the vir genes harbored by Agrobacterium Ti plasmids, other, chromosomally-borne virulence controlling genes (termed chv genes) are known to control certain aspects of the interactions of Agrobacterium cells and plant cells, and thus affect the overall plant transformation frequency (Pan et al., 1995, Molec. Microbiol. 17:259-269). Several of the chromosomally-borne genes required for virulence and attachment are grouped together in a chromosomal locus spanning 29 kilobases (Matthysse et al., 2000, Biochim. Biophys. Acta 1490:208-212).

In addition to numerous technologies for transforming plants, the type of tissue which is contacted with the foreign genes may vary as well. Such tissue may include, but is not limited to, embryogenic tissue, callus tissue types I and II, hypocotyl, and meristem. Almost all plant tissues may be transformed during dedifferentiation using appropriate techniques within the skill of an artisan. One skilled in the field of plant transformation will understand that multiple methodologies are available for the production of transformed plants, and that they may be modified and specialized to accommodate biological differences between various host plant species. Plant explants (for example, pieces of leaf, segments of stalk, meristems, roots, but also protoplasts or suspension-cultivated cells) can advantageously be cultivated with Agrobacterium tumefaciens or Agrobacterium rhizogenes for the transfer of the DNA into the plant cell.

Callus Cultures

Plant tissue cultures may advantageously be cultivated with Agrobacterium tumefaciens or Agrobacterium rhizogenes for the transfer of the DNA into the plant cell, and are generally initiated from sterile pieces of a whole plant that may consist of pieces of organs, such as leaves or roots, or maybe specific cell types, such as pollen or endosperm. Many features of the explant are known to affect the efficiency of culture initiation. It is thought that any plant tissue can be used as an explant, if the correct conditions are found. Generally, younger, more rapidly growing tissue (or tissue at an early stage of development) is most effective. Explants cultured on the appropriate medium can give rise to an unorganized, growing, and dividing mass of cells (callus). In culture, callus can be maintained more or less indefinitely, provided that it is subcultured on to fresh medium periodically. During callus formation, there is some degree of de-differentiation, both in morphology (a callus is usually composed of unspecialized parenchyma cells) and metabolism.

Callus cultures are extremely important in plant biotechnology. Manipulation of the plant hormone ratios in the culture medium can lead to the development of shoots, roots, or somatic embryos from which whole plants can subsequently be produced (regeneration). Callus cultures can also be used to initiate cell suspensions, which are used in a variety of ways in plant transformation studies.

Cell Suspension Cultures

Callus cultures, broadly speaking, fall into one of two categories: compact or friable. In compact callus, the cells are densely aggregated, whereas in friable callus, the cells are only loosely associated with each other and the callus becomes soft and breaks apart easily. Friable callus provides the inoculum to form cell-suspension cultures. Explants from some plant species or particular cell types tend not to form friable callus, making it difficult to initiate cell suspension. The friability of the callus can sometimes be improved by manipulating the medium components, by repeated subculturing, or by culturing it on semi-solid medium (medium with a low concentration of gelling agent). When friable callus is placed into a liquid medium and then agitated, single cells and/or small clumps of cells are released into the medium. Under the correct conditions, these released cells continue to grow and divide, eventually producing a cell-suspension culture. Cell suspensions can be maintained relatively simply as batch cultures in conical flasks and are propagated by repeated subculturing into fresh medium. After subculture, the cells divide and the biomass of the culture increases in a characteristic fashion. Cell suspension cultures may advantageously be cultivated with Agrobacterium tumefaciens or Agrobacterium rhizogenes for the transfer of the DNA into the plant cell.

Shoot Tip and Meristem Culture

The tips of shoots (which contain the shoot apical meristem) can be cultured in vitro, producing clumps of shoots from either axillary or adventitious buds and may advantageously be cultivated with Agrobacterium tumefaciens or Agrobacterium rhizogenes for the transfer of the DNA into the plant cell. Shoot meristem cultures are used for cereal regeneration (seedlings can be used as donor material).

Embryo Culture

Embryos can be used as explants to generate callus cultures or somatic embryos. Both immature and mature embryos can be used as explants. Immature, embryo-derived embryogenic callus is a tissue used in monocotyledon plant regeneration and may advantageously be cultivated with Agrobacterium tumefaciens for the transfer of the DNA into the plant cell. Immature embryos are an intact tissue that is capable of cell division to give rise to callus cells that can differentiate to produce tissues and organs of a whole plant. Immature embryos can be obtained from the fertilized ears of a mature maize plant, for example, from plants pollinated using the methods of Neuffer et al. (1982, Growing maize for genetic purposes. In: Maize for Biological Research. W. F. Sheridan, Ed. UNIVERSITY PRESS, University of North Dakota, Grand Forks, N. Dak.). Exemplary methods for isolating immature embryos from maize are described by Green and Phillips (Crop Sci. 15:417-421 (1976)). Immature embryos are preferably isolated from the developing ear using aseptic methods and are held in sterile medium until use. The use of Agrobacterium in transformation of immature embryos is disclosed by Sidorov & Duncan, (2009, Methods in Molecular Biology: Transgenic Maize, vol. 526 Chapter 4, M. Paul Scott (Ed.)) and in U.S. Pat. No. 5,981,840.

Microspore Culture

Haploid tissue can be cultured in vitro by using pollen or anthers as an explant and may advantageously be cultivated with Agrobacterium tumefaciens for the transfer of the DNA into the plant cell. Both callus and embryos can be produced from pollen. Two approaches can be taken to produce cultures in vitro from haploid tissue. In the first, anthers (somatic tissue that surrounds and contains the pollen) are cultured on solid medium. Pollen-derived embryos are subsequently produced via dehiscence of the mature anthers. The dehiscence of the anther depends both on its isolation at the correct stage and on the correct culture conditions. In some species, the reliance on natural dehiscence can be circumvented by cutting the wall of the anther. In the second method, anthers are cultured in liquid medium, and pollen released from the anthers can be induced to form embryos. Immature pollen can also be extracted from developing anthers and cultured directly.

Many of the cereals (rice, wheat, barley, and maize) require medium supplemented with plant growth regulators for pollen or anther culture. Regeneration from microspore explants can be obtained by direct embryogenesis, or via a callus stage and subsequent embryogenesis.

Haploid tissue cultures can also be initiated from the female gametophyte (the ovule). In some cases, this is a more efficient method than using pollen or anthers.

Plants obtained from haploid cultures may not be haploid. This can be a consequence of chromosome doubling during the culture period. Chromosome doubling (which may be induced by treatment with chemicals such as colchicine) may be an advantage, as in many cases haploid plants are not the desired outcome of regeneration from haploid tissues. Such plants are often referred to as di-haploids, because they contain two copies of the same haploid genome.

Following transformation of any of the aforementioned plant materials by cultivation with Agrobacterium tumefaciens for the transfer of the DNA into the plant cell, whole plants may then be regenerated from the infected plant material following placement in suitable growth conditions and culture medium, which may contain antibiotics or herbicides for selection of the transformed plant cells. The plants so obtained can then be tested for the presence of the inserted DNA.

Cell transformation (including plant cell transformation) may involve the construction of an expression vector which will function in a particular cell. Such a vector may comprise DNA that includes a gene under control of, or operatively linked to, a regulatory element (for example, a promoter). The expression vector may contain one or more such operably-linked gene/regulatory element combinations. The vector(s) may be in the form of a plasmid and can be used alone or in combination with other plasmids to provide transformed cells using transformation methods as described herein to incorporate transgene(s) into the genetic material of a plant cell.

Plant cell expression vectors may include at least one genetic marker, operably linked to a regulatory element (a promoter, for example) that allows transformed cells containing the marker to be either recovered by negative selection (i.e., inhibiting growth of cells that do not contain the selectable marker gene) or by positive selection (i.e., screening for the product encoded by the genetic marker). Many selectable marker genes suitable for plant transformation are well known in the transformation arts and include, for example, genes that code for enzymes that metabolically detoxify a selective chemical agent which may be an antibiotic or an herbicide, or genes that encode an altered target which may be insensitive to the inhibitor. A few positive selection methods are also known in the art. The individually employed selectable marker gene may accordingly permit the selection of transformed cells while the growth of cells that do not contain the inserted DNA can be suppressed by the selective compound. The preference for a particular selectable marker gene is at the discretion of the artisan, but any of the following selectable markers may be used, as well as any other gene not listed herein which could function as a selectable marker. Examples of selectable markers include, but are not limited, to resistance or tolerance to Kanamycin, G418, Hygromycin, Bleomycin, Methotrexate, Phosphinothricin (Bialaphos), Glyphosate, Imidazolinones, Sulfonylureas and Triazolopyrimidine herbicides, such as Chlorosulfuron, Bromoxynil, and DALAPON.

In addition to a selectable marker, it may be desirable to use a reporter gene. In some instances a reporter gene may be used without a selectable marker. Reporter genes are genes which typically do not provide a growth advantage to the recipient organism or tissue. The reporter gene typically encodes for a protein which provides for some phenotypic change or enzymatic property. Suitable reporter genes include, but are not limited to, those that encode beta-glucuronidase (GUS), firefly luciferase, or fluorescent proteins such as green fluorescent protein (GFP) or yellow fluorescent protein (YFP, essentially as disclosed in U.S. Pat. No. 7,951,923).

Regardless of transformation technique utilized, the foreign gene can be incorporated into a gene transfer vector adapted to express the foreign gene in the plant cell by including in the vector a plant promoter. In addition to plant promoters, promoters from a variety of sources can be used efficiently in plant cells to express foreign genes. For example, promoters of bacterial origin, such as the octopine synthase promoter, the nopaline synthase promoter, the mannopine synthase promoter; promoters of viral origin, such as the 35S and 19S promoters of cauliflower mosaic virus (CaMV), a promoter from sugarcane bacilliform virus, and the like may be used. Plant-derived promoters include, but are not limited to ribulose-1,6-bisphosphate (RUBP) carboxylase small subunit (ssu), beta-conglycinin promoter, phaseolin promoter, ADH (alcohol dehydrogenase) promoter, heat-shock promoters, ADF (actin depolymerization factor) promoter, and tissue specific promoters. Promoters may also contain certain enhancer sequence elements that may improve the transcription efficiency. Typical enhancers include, but are not limited to, alcohol dehydrogenase 1 (ADH1) intron 1 and ADH1-intron 6. Constitutive promoters may be used. Constitutive promoters direct continuous gene expression in nearly all cells types and at nearly all times (e.g. actin, ubiquitin, CaMV 35S). Tissue specific promoters are responsible for gene expression in specific cell or tissue types, such as the leaves or seeds Examples of other promoters that may be used include those that are active during a certain stage of the plant's development, as well as active in specific plant tissues and organs. Examples of such promoters include, but are not limited to, promoters that are root specific, pollen-specific, embryo specific, corn silk specific, cotton fiber specific, seed endosperm specific, and phloem specific.

Under certain circumstances, it may be desirable to use an inducible promoter. An inducible promoter is responsible for expression of genes in response to a specific signal, such as: physical stimulus (e.g. heat shock genes); light (e.g. Ribulose-bis-phosphate 1,5 carboxylase); hormone (e.g. glucocorticoid); antibiotic (e.g. Tetracycline); metabolites; and stress (e.g. drought). Other desirable transcription and translation elements that function in plants also may be used, such as, for example, 5′ untranslated leader sequences, and 3′ RNA transcription termination and poly-adenylate addition signal sequences. Any suitable plant-specific gene transfer vector known to the art may be used.

Transgenic crops containing insect resistance (IR) traits are prevalent in corn and cotton plants throughout North America, and usage of these traits is expanding globally. Commercial transgenic crops combining IR and herbicide tolerance (HT) traits have been developed by multiple seed companies. These include combinations of IR traits conferred by Bacillus thuringiensis (B.t.) insecticidal proteins and HT traits such as tolerance to Acetolactate Synthase (ALS) inhibitors such as Sulfonylureas, Imidazolinones, Triazolopyrimidine, Sulfonanilides, and the like, Glutamine Synthetase (GS) inhibitors such as Bialaphos, Glufosinate, and the like, 4-HydroxyPhenylPyruvate Dioxygenase (HPPD) inhibitors such as Mesotrione, Isoxaflutole, and the like, 5-EnolPyruvylShikimate-3-Phosphate Synthase (EPSPS) inhibitors such as Glyphosate and the like, and Acetyl-Coenzyme A Carboxylase (ACCase) inhibitors such as Haloxyfop, Quizalofop, Diclofop, and the like. Other examples are known in which transgenically provided proteins provide plant tolerance to herbicide chemical classes such as phenoxy acids herbicides and pyridyloxyacetates auxin herbicides (see WO 2007/053482 A2), or phenoxy acids herbicides and aryloxyphenoxypropionates herbicides (see WO 2005/107437A1). The ability to control multiple pest problems through IR traits is a valuable commercial product concept, and the convenience of this product concept is enhanced if insect control traits and weed control traits are combined in the same plant. Further, improved value may be obtained via single plant combinations of IR traits conferred by a B.t. insecticidal protein with one or more additional HT traits such as those mentioned above, plus one or more additional input traits (e.g. other insect resistance conferred by B.t.-derived or other insecticidal proteins, insect resistance conferred by mechanisms such as RNAi and the like, disease resistance, stress tolerance, improved nitrogen utilization, and the like), or output traits (e.g. high oils content, healthy oil composition, nutritional improvement, and the like). Such combinations may be obtained either through conventional breeding (e.g. breeding stack) or jointly as a novel transformation event involving the simultaneous introduction of multiple genes (e.g. molecular stack). Benefits include the ability to manage insect pests and improved weed control in a crop plant that provides secondary benefits to the producer and/or the consumer. Thus, the methods of this disclosure can be used to provide transformed plants with combinations of traits that comprise a complete agronomic package of improved crop quality with the ability to flexibly and cost effectively control any number of agronomic issues.

### SUMMARY

Methods for plant cell transformation are described. These methods include exposing the plant cells to Agrobacterium cells in a liquid medium containing a surfactant. The Agrobacterium cells can be scraped from a solid medium or grown in a liquid growth medium prior to being suspended in the liquid medium containing the surfactant. The concentration of surfactant can be in the range of 0.001 weight percent to 0.08 weight percent. The surfactant can be a non-ionic trisiloxane surfactant and more than one surfactant can be used. The plant cells can be maize cells. The plant cells can be exposed to continuous light after exposure to the Agrobacterium cells.

### DESCRIPTION OF DRAWINGS

FIG. 1 is a bar graph showing the enhancement of maize immature embryo transformation when the surfactant BREAK-THRU® S 233 is added to the Infection Medium used to create a suspension of Agrobacterium cells (harboring plasmid pEPS1083) prior to co-cultivation.

FIG. 2 is a bar graph showing the enhancement of maize immature embryo transformation when the surfactant BREAK-THRU® S 233 is added to the Infection Medium used to create a suspension of Agrobacterium cells prior to co-cultivation. The Plasmids used for each experiment shown in FIG. 2 include: Experiment 1=pEPS1053; GOI=IPT, selectable marker=aad1. Experiment 2=pEPS1038; GOI=GF14, selectable marker=aad1. Experiment 3 and Experiment 4=pEPS1027; no GOI, selectable marker=aad1.

### DETAILED DESCRIPTION

Methods to increase transformation frequency in plants when using Agrobacterium are described. The methods include exposing plant cells to Agrobacterium cells in a liquid medium containing a surfactant. Some methods include exposing the plant cells to continuous light after exposure to the Agrobacterium cells. Examples of plants useful with these methods include maize plants and immature maize embryos.

Strains of Agrobacterium differ from one another in their ability to transform plant cells of various species. Regardless of the particular combination of Agrobacterium strain/host plant considered, Agrobacterium acts through attachment to the host cell during transformation. See McCullen and Binns, 2006, Ann. Rev. Cell. Dev. Biol. 22:101-127; and Citovsky et al., 2007, Cell. Microbiol. 9:9-20. For this reason, methods that enhance binding of Agrobacterium cells to plant cells, such as those disclosed herein using surfactants, may produce increases in transformation efficiency. Enhancing the binding of Agrobacterium cells to plant cells is different for different species and tissue types as different plant species, and further, different tissues of a plant of a single species, can differ in chemical and biochemical composition of their cell walls. Further, such differences may also vary during different developmental stages of a single plant tissue.

Additionally different genera and species of bacteria, and indeed, different strains of a bacterial species, often differ in chemical and biochemical composition of their cell walls, and these differences can change during the bacterial growth cycle. Increases in plant transformation efficiencies by the methods disclosed herein thus may result from the ability of surfactants to decrease hydrophobic repulsive interactions between Agrobacterium cell walls and plant cell walls, and thus allow intimate cell-cell interactions to occur.

One may therefore utilize the chemical differences between different surfactant agents to promote cell-cell interactions between cells of different Agrobacterium strains (and different growth phases of such cells) and cells and tissues of different host plants during various phases of culture of the plant tissues so that enhanced transformation efficiencies may be observed.

Surfactants belong to several chemical classes, and one skilled in the field of plant transformation will understand that different chemical classes of surfactants may be used to enhance plant transformation efficiency with different plant hosts. Examples of surfactants from these chemical classes useful with the methods disclosed herein include adjuvants, non-ionic surfactants, anionic surfactants, oil based surfactants, amphoteric surfactants, and polymeric surfactants. An example of a preferred surfactant useful with the methods described herein is a non-ionic trisiloxane surfactant such as BREAK-THRU® 5233 from Evonik Industries (Essen, Germany). Examples of further preferred surfactants useful with the methods described herein include trisiloxane alkoxylates, ethoxylated soybean oils, alcohol ethoxylate C-13s, C₁₂-C₁₄-alkyldimethyl betaines, and di-sec-butylphenol ethylene oxide-propylene oxide block co-polymers. Table 1 presents an non-limiting list of surfactants of various chemical type that may be used to practice the methods described herein.

TABLE 1
Surfactants groupings, commercial names and chemical action/class.

| | Trade | Chemistry | |
|---|---|---|---|
| Group | Name | Class | Supplier |
| Adjuvant | BREAK-THRU ® | Polyether-modified | EVONIK |
| | S 240 | polysiloxane | INDUSTRIES AG |
| | | | (Essen, Germany) |
| | BREAK-THRU ® | Organo-modified | EVONIK |
| | S 243 | polysiloxane | INDUSTRIES AG |
| | SILWET ® HS 429 | Hydrolytically stable | GE SILICONES |
| | | silicone | (Friendly, WV) |
| | SILWET ® 618 | Trisiloxane alkoxylate | GE SILICONES. |
| | SILWET ® 625 | Trisiloxane alkoxylate | GE SILICONES. |
| | SILWET ® HS 312 | Hydrolytically stable silicone | GE SILICONES |
| | SILWET ® L-77 | Trisiloxane alkoxylate | GE SILICONES |
| | HI-WETT ® | Blend of organosilicone & | ELLIOTT |
| | | other organic fluids. | CHEMICALS LTD. |
| | | | (Aukland, NZ) |
| Non-ionic | SYLGARD ® 309 | Non-ionic silicone surfactant | DOW CORNING |
| surfactant | | | (Midland, MI) |
| | AGRIMUL ® PG 2069 | Alkylpolyglucoside | HENKEL |
| | | | CORPORATION |
| | | | (Berkeley, CA) |
| | TRYCOL ® 5941 | Alcohol ethoxylate C-13 | MONSON |
| | | (9 mole EO*) | COMPANIES, INC |
| | | | (Leominster, MA) |
| | MAKON ® TD-6 | Alcohol ethoxylate C-13 | STEPAN COMPANY |
| | | (6 mole EO) | (Northfield, IL) |
| | MAKON ® TD-12 | Alcohol ethoxylate C-13 | STEPAN COMPANY |
| | | (12 mole EO) | |
| | TRYCOL ® 5993-A | Alcohol ethoxylate C-13 | MONSON |
| | | (3 mole EO) | COMPANIES, INC |
| | PREFERENCE ® | Non-ionic surfactant and | AGRILIANCE LLC |
| | | anti-foaming agent | (Inver Grove Heights, |
| | | | MN) |
| | PLURONIC ® P105 | Ethylene Oxide/Propylene | BASF |
| | | Oxide Block Copolymer | CORPORATION |
| | | | (Cincinnati, OH) |
| Anionic | NANSA ® HS 90/S | Sodium alkyl benzene | HUNTSMAN |
| surfactant | | sulphonate | CORPORATION |
| | | | (The Woodlands, TX) |
| Oil-based | SLS (no trade name) | Sodium lauryl sulfate | SIGMA-ALDRICH |
| | | | (St. Louis, MO) |
| | EMERY ®/EMGARD ® | Methyloleate/surfactants | HENKEL |
| | | | CORPORATION |
| | AGNIQUE ® SBO-10 | Ethoxylated soybean oil | BASF |
| | | (10 mole POE**) | CORPORATION |
| | UPTAKE ® | di-sec-butylphenol ethylene | DOW |
| | | oxide-propylene oxide block | AGROSCIENCES |
| | | co-polymer | (Indianapolis, IN) |
| | LI-700 ® | soy-oil derived, non-ionic | LOVELAND |
| | | penetrating surfactant | PRODUCTS INC. |
| | | | (Greeley, CO) |
| Amphoteric | AMMONYX ® (C-12) | Amine oxide | STEPAN COMPANY |
| surfactant | ADSEE ® AB-650 | 100% Alkoxylated fatty | AKZONOBEL |
| | | amine + wetting agent + | SURFACE |
| | | buffer | CHEMISTRY LLC |
| | | | (Chicago, IL) |
| | BREAK-THRU ® G | Fatty acid amido alkyl | EVONIK |
| | 850 | betaine | INDUSTRIES AG |
| | GERONOL ® CF/AS | Betaines, C12-14- | RHODIA INC |
| | 30 | alkyldimethyl | (Cranberry, NJ) |
| Polymeric | BORRESPERSE ® NA | Lignosulfonate | ORKLA INDIA PVT |
| | | | LTD |
| | | | (Vashi, India) |
| | MORWET ® D-425 | Alkylnaphthalene sulfonate | MONSON |
| | | condensate | COMPANIES, INC |
| | ATLOX ™ 4913 | Non-ionic comb polymer | CRODA WEST |
| | | | Chino Hills, CA |
| | METASPERSE ™ | Modified styrene acrylic | CRODA WEST |
| | 500L | polymer | |
| | AGRIMER ® AL10 | Alkylated vinylpyrrolidone | INTERNATIONAL |
| | | copolymers | SPECIALTY |
| | | | PRODUCTS |
| | | | (Wayne, NJ) |
| | CEVOL ® 205 | Polyvinyl alcohol | CELANESE LTD. |
| | | | (Dallas, TX) |
| | ALCOSPERSE ® 725 | Hydrophobically modified | AKZONOBEL |
| | | polyacrylate | SURFACE |
| | | | CHEMISTRY LLC |
| | TACTIC ™ | 1,2-propanediol, Alcohol | LOVELAND |
| | | ethoxylate, silicone polyether | PRODUCTS INC. |
| | | copolymer | |

*EO = moles of ethylene oxide reacted with a particular hydrophobe
**POE = moles of propylene oxide reacted with a particular hydrophobe

The methods disclosed herein utilize the transformation-enhancing properties of surfactants to dramatically increase transformation efficiency in plants such as immature maize embryos by Agrobacterium (e.g., Agrobacterium tumefaciens). The surfactants used with the methods described herein are selected, as suggested above, based upon the ability to promote cell-cell interactions that will enhance transformation efficiency. The concentration of surfactant in the liquid medium can be 0.001 weight percent to 0.08 weight percent, 0.001 weight percent to 0.07 weight percent, 0.001 weight percent to 0.06 weight percent, 0.001 weight percent to 0.05 weight percent, 0.001 weight percent to 0.04 weight percent, 0.001 weight percent to 0.035 weight percent, 0.001 weight percent to 0.03 weight percent, 0.001 weight percent to 0.025 weight percent, 0.001 weight percent to 0.02 weight percent, 0.001 weight percent to 0.015 weight percent, 0.001 weight percent to 0.01 weight percent, or 0.005 weight percent to 0.01 weight percent.

One or more additional surfactants can also be used with the methods described herein. As indicated, the transformation efficiency is dependent on a variety of factors including plant species and tissue-type and Agrobacterium strain. Given the variety of interactions involved, a system of two or more surfactants can provide enhanced transformation efficiency. The additional surfactants used in a system of two or more surfactants can be selected, for example, from Table 1.

The methods described herein are broadly applicable to a variety of plant species and varieties including monocotyledons and dicotyledons. Crops of interest include but are not limited to maize, rice, soybeans, canola, sunflower, alfalfa, sorghum, wheat, cotton, peanuts, tomatoes, potatoes, and the like. The methods herein can be used with cells at various stages of development, e.g., immature embryos. Thus, the methods described herein can be used to transform maize immature embryos. The size of immature embryos used in the methods described herein can vary. For example, immature embryos can be greater than or equal to 1.5 mm and less than or equal to 2.5 mm in length.

The external environment the cells are maintained in after exposure to Agrobacterium according to the methods described herein can be controlled. For example, temperature, pH, and other components of growth medium the cells are placed upon after transformation according to the methods described herein can be varied and are generally well known to those of skill in the art. One of those variables is exposure to light. The methods described herein can include exposing the plant cells to common 18 hour light/6 hour dark protocols or alternatively to continuous light after exposure to the Agrobacterium cells. For example, cells treated according to the methods described herein can be exposed to 24-hour white fluorescent light conditions for weeks after treatment, e.g., until the regeneration and plantlet isolation stages of plant preparation.

An additional method includes preparing a liquid medium containing a surfactant, suspending Agrobacterium cells in the liquid medium, and exposing plant cells to the Agrobacterium cells in the liquid medium containing the surfactant. The Agrobacterium cells can be scraped from a solid medium prior to being suspended in the liquid medium containing a surfactant. Additionally, the Agrobacterium cells can be grown in a liquid growth medium prior to being suspended in the liquid medium containing a surfactant.

Protocols and methods for transforming plants using Agrobacterium are well known to those of skill in the art of molecular biology. Any types of methods known for the use of Agrobacterium in transforming plants can be used with the methods described herein. The examples below provide embodiments of methods demonstrating the effectiveness of the methods described herein, but are not intended to be limitations on the scope of the claims.

All patents, patent applications, provisional applications, and publications referred to or cited herein are incorporated by reference in their entirety to the extent they are not inconsistent with the explicit teachings of this specification.

### EXAMPLES

The following examples illustrate procedures for practicing the claims. The examples and embodiments described herein are for illustrative purposes only and various modifications or changes in light thereof will be suggested to persons skilled in the art and are to be included within the spirit and scope of the claims. All percentages are by weight and all solvent mixture proportions are by volume unless otherwise noted. All temperatures are in degrees Celsius.

### Example 1

### Agrobacterium Transformation for Generation of Superbinary Vectors

The Agrobacterium superbinary system is conveniently used for transformation of monocot plant hosts. Methodologies for constructing and validating superbinary vectors are well disclosed and incorporated herein by reference (Operating Manual for Plasmid pSB1, Version 3.1, available from Japan Tobacco, Inc., Tokyo, Japan). Standard molecular biological and microbiological methods were used to generate superbinary plasmids. Verification/validation of the structure of the superbinary plasmid was done using methodologies as suggested in the Operating Manual for Plasmid pSB1.

Agrobacterium strains harboring various superbinary plasmids were used in this work. All these plasmids contained, as the selectable marker/herbicide tolerance gene, the coding sequence (CDS) for the AAD1 protein (U.S. Pat. No. 7,838,733), whose expression was under the transcriptional control of a rice actin1 promoter and associated intron 1, essentially as disclosed in U.S. Pat. No. 5,641,876 and GENBANK™ Accession No. EU155408.1. Termination of transcription and polyadenylation of the aad1 mRNAs were determined by a maize lipase 3′UTR, essentially as disclosed as bases 921 to 1277 of GENBANK™ Accession No. gb|L35913.1|MZELIPASE and in U.S. Pat. No. 7,179,902. In addition, the superbinary plasmids harbored a gene whose expression was not expected to affect transformation frequency. In particular, in plasmid pEPS1083, a CDS encoding a YFP protein (essentially as disclosed in U.S. Pat. No. 7,951,923) (transcription of which was controlled by a maize ubiquitin 1 promoter with associated intron 1; U.S. Pat. No. 5,510,474), and whose mRNAs were terminated by a maize Per5 3′UTR (U.S. Pat. No. 6,384,207)) was advantageously used as a visual marker to monitor transformation and determine relative transformation efficiencies. Other superbinary plasmids used to exemplify the methods disclosed here (plasmids pEPS1013, pEPS1018, pEPS1028, pEPS1036, pEPS1038, pEPS1059, pEPS1064, pEPS1066, pEPS1068, pEPS6004, and pEPS6008) harbored a CDS encoding a Dow AgroSciences proprietary protein, expression of which was controlled by the same transcription/termination elements as were used for the YFP CDS.

Expression of YFP was used to measure the efficiency of transformation in some experiments. Transformation efficiency percentages were calculated as the number of calli that displayed expression of YFP, divided by the number of treated embryos, times 100. YFP expression was measured by visual observation using either an Olympus SZX12 (Olympus America Inc.; Center Valley, Pa.) or a Leica M165FC (Leica Microsystems Inc.; Buffalo Grove, Ill.) fluorescent microscope, with YFP filters covering the ranges for excitation at 514 nm and emission measured at 527 nm.

In other experiments that employed Agrobacterium strains harboring superbinary plasmids lacking the YFP gene, transformation efficiencies were calculated following TAQMAN® analysis (Life Technologies; Carlsbad, Calif.) of progeny plants produced from embryos that were selected by means of resistance to Haloxyfop. The TAQMAN® components used were specific for the aad1 coding region. Transformation efficiencies were calculated from the number of TAQMAN®-positive events determined, divided by the number of treated embryos, times 100. An “event” for these purposes was considered to be an embryo that produced one or more TAQMAN®-verified plant(s). An individual embryo was considered to be one event regardless of how many plants it may have produced.

### Example 2

### Transformation of Maize by Agrobacterium Strains (Transformation Protocol 1)

The basic work flow is summarized as follows. Embryos are extracted from immature ears of corn at the developmental stage at which the young embryos are about 1.4 to 1.9 mm in length. When different transformation conditions are to be compared, approximately equal numbers of embryos isolated from a single ear are divided amongst all the treatments. The embryos are incubated with a suspension containing Agrobacterium cells and surfactant (or not, for comparison), then are moved to solid-medium plates and co-cultivation is allowed for 3 to 5 days. The treated embryos are transferred onto a medium containing antibiotics (for the suppression and killing of the Agrobacterium cells) and compounds for the selective isolation of genetically transformed corn tissues and plants. The corn tissue (usually, but not limited to, callus) is grown on selection medium until plants are regenerated. These plants are tested to confirm their genetic transformation and those having a desired modification are grown to maturity for seed production.

Immature Embryo Production

Seeds from a B104 inbred were planted into 4-gallon-pots containing SUNSHINE CUSTOM BLEND 160 (SUN GRO HORTICULTURE; Bellevue, Wash.). The plants were grown in a greenhouse using a combination of high pressure sodium and metal halide lamps with a 16:8 hour Light:Dark photoperiod. To obtain immature embryos for transformation, controlled sib-pollinations were performed. Immature embryos were isolated at 10 to 13 days post-pollination when embryos were approximately 1.4 to 1.9 mm in size. Maize ears were surface sterilized after removing the husks and silks by immersing in 50% commercial bleach (CLOROX®, 5.25% sodium hypochlorite) with TWEEN®-20 (1 or 2 drops per 500 mL) for 10 minutes and triple-rinsed with sterile water.

Immature embryos were aseptically isolated directly into a micro centrifuge tube containing 2 mL of Infection Medium with suspended Agrobacterium cells, and surfactant as appropriate. The embryos were incubated with the suspension of Agrobacterium cells, containing surfactant (or not, for control experiments), for 5-30 minutes.

A suspension of Agrobacterium cells containing a superbinary vector was prepared by first growing the cells as a lawn for 4 days at 25°, or 3 days at 28°, on solid agar plates containing YEP (gm/L: Yeast Extract, 5; Peptone, 10; NaCl, 5; agar, 15) with 50 mg/L Spectinomycin; 10 mg/L Rifampicin; and 50 mg/L Streptomycin. (In some experiments, the Agrobacterium cells were grown on solid LB medium (SIGMA ALDRICH; St. Louis, Mo.) 20 gm/L, with antibiotics as above.) This culture was streaked from a single colony isolate established under the same conditions. One or two loopfuls of cells were scraped from the lawn, then uniformly resuspended (by gently pipetting up and down) in Infection Medium (IfM) to an optical density at 600 nm (OD₆₀₀) of 0.35 to 0.45. Infection Medium contains: 4.33 gm/L MS salts; 1×ISU Modified MS Vitamins; 68.4 gm/L sucrose; 36 gm/L glucose; 700 mg/L L-proline; 3.3 mg/L Dicamba-KOH; and 100 μM acetosyringone (prepared in DMSO); at pH 5.2. Depending upon the experiment, an appropriate amount of surfactant solution (e.g. BREAK-THRU® S 233 at 0.01% final concentration) was added to the Infection Medium after suspending the cells.

The Agrobacterium and embryo solution was incubated for 5 to 30 minutes at room temperature, and then the embryos were transferred to Co-cultivation Medium, which contained 4.33 gm/L MS salts; 1×ISU Modified MS Vitamins; 30 gm/L sucrose; 700 mg/L L-proline; 100 mg/L myo-inositol; 3.3 mg/L Dicamba-KOH; 100 mg/L Casein Enzymatic Hydrolysate; 15 mg/L AgNO₃; 100 μM acetosyringone; and 3 gm/L GELZAN™; at pH 5.8. Co-cultivation incubation was for 3 to 4 days at 25° under 24-hour white fluorescent light (approximately 50 μEm⁻²s⁻¹).

Resting and Selection

After co-cultivation, the embryos (36 embryos/plate) were carefully transferred to fresh non-selection Resting Medium containing 4.33 gm/L MS salts; 1×ISU Modified MS Vitamins; 30 gm/L sucrose; 700 mg/L L-proline; 3.3 mg/L Dicamba in KOH; 100 mg/L myo-inositol; 100 mg/L Casein Enzymatic Hydrolysate; 15 mg/L AgNO₃; 0.5 gm/L MES; 250 mg/L Carbenicillin; and 2.3 gm/L GELZAN™; at pH 5.8. Incubation was continued for 7 days at 28° in 24-hour white fluorescent light (approximately 50 μEm⁻²s⁻¹).

Following the 7 day resting period, the embryos were transferred to Selection Medium. For selection of maize tissues transformed with a superbinary plasmid containing a plant expressible aad1 selectable marker gene, the embryos (36/plate) were first transferred to Selection Medium I, which comprised Resting Medium (above) containing 100 nM R-Haloxyfop acid (0.0362 mg/L). The embryos were incubated for 1 week (28°; continuous light), and then transferred to Selection Medium II which comprised Resting Medium with 500 nM R-Haloxyfop acid (0.1810 mg/L), on which they were incubated under continuous light for an additional 7 days. At this time they were moved to fresh Selection Medium II and incubation was continued as above for an additional week.

Those skilled in the art of maize transformation will understand that other methods of selection of transformed plants are available when other plant expressible selectable marker genes (e.g. herbicide tolerance genes) are used.

Pre-Regeneration

Following the selection process, cultures were transferred to Pre-regeneration Medium containing 4.33 gm/L MS salts; 1×ISU Modified MS Vitamins; 45 gm/L sucrose; 350 mg/L L-proline; 100 mg/L myo-inositol; 50 mg/L Casein Enzymatic Hydrolysate; 1.0 mg/L AgNO₃; 0.25 gm/L MES; 0.5 mg/L naphthaleneacetic acid in NaOH; 2.5 mg/L abscisic acid in ethanol; 1 mg/L 6-benzylaminopurine; 250 mg/L Carbenicillin; 2.5 gm/L GELZAN™; and 500 nM R-Haloxyfop acid; at pH 5.8. Incubation was continued for 7 days at 28° under continuous white fluorescent light as above.

Regeneration and Plantlet Isolation

For regeneration, the cultures were transferred to Regeneration Medium I containing 4.33 gm/L MS salts; 1×ISU Modified MS Vitamins; 60 gm/L sucrose; 100 mg/L myo-inositol; 125 mg/L Carbenicillin; 2.5 gm/L GELZAN™; and 500 nM R-Haloxyfop acid; at pH 5.8 and plantlets were allowed to generate and grow at 28° under continuous white fluorescent light for up to 3 weeks.

When plantlets reached a suitable growth stage, they were excised with a forceps and scalpel and transferred to Regeneration Medium II containing 4.33 gm/L MS salts; 1×ISU Modified MS Vitamins; 30 gm/L sucrose; 100 mg/L myo-inositol; 3.0 gm/L GELZAN™; at pH 5.8; and incubated at 28° under continuous white fluorescent light as above to allow for further growth and development of the shoot and roots.

Seed Production

Plants were transplanted into METRO-MIX 360 soilless growing medium (SUN GRO HORTICULTURE; BELLEVUE, WA) and hardened-off in a growth room. Plants were then transplanted into SUNSHINE CUSTOM BLEND 160 soil mixture and grown to flowering in the greenhouse. Controlled pollinations for seed production were conducted.

### Example 3

### Transformation of Maize by Agrobacterium Strains (Transformation Protocol 2)

The basic work flow is summarized as follows. Embryos are extracted from immature ears of corn at the developmental stage at which the young embryos are about 1.8 to 2.4 mm in length. When different transformation conditions are to be compared, approximately equal numbers of embryos isolated from a single ear are divided amongst all the treatments. The embryos are incubated with a suspension containing Agrobacterium cells and surfactant (or not, for comparison), then are moved to solid-medium plates and co-cultivation is allowed for 1 to 4 days. The treated embryos are transferred onto a medium containing antibiotics (for the suppression and killing of the Agrobacterium cells) and compounds for the selective isolation of genetically transformed corn tissues and plants. The corn tissue (usually, but not limited to, callus) is grown on selection medium until plants are regenerated. These plants are tested to confirm their genetic transformation and those having a desired modification are grown to maturity for seed production.

Immature Embryo Production

Seeds from maize inbred line B104 (an Iowa State variety commercially released in the early 1980's) were planted into 4-gallon-pots containing SUNSHINE CUSTOM BLEND 160 (SUN GRO HORTICULTURE; Bellevue, Wash.). The plants were grown in a greenhouse using a combination of high pressure sodium and metal halide lamps with a 16:8 hour Light:Dark photoperiod. To obtain immature embryos for transformation, controlled sib-pollinations were performed. Immature embryos were isolated at 10 to 13 days post-pollination when embryos were approximately 1.8 to 2.4 mm in size. Maize ears were surface sterilized after removing the husks and silks by immersing in 50% commercial bleach (CLOROX®, 6.15% sodium hypochlorite) with TWEEN®-20 (1 or 2 drops per 500 mL) for 10 minutes and triple-rinsed with sterile water.

Alternatively, maize ears can be surface sterilized by thorough spraying with a freshly prepared solution of 70% ethanol until the ear is completely soaked. Prior to use, the ear is allowed to air dry for half an hour in a sterile transfer hood to allow the ethanol solution to completely evaporate.

Immature embryos were aseptically isolated directly into a micro centrifuge tube containing 2 mL of Inoculation Medium with suspended Agrobacterium cells, and surfactant as appropriate. The embryos were incubated with the suspension of Agrobacterium cells, containing surfactant (or not, for control experiments), for 5-30 minutes.

A suspension of Agrobacterium cells containing a superbinary vector was prepared by first growing the cells in 125 mL (in 500 mL baffled flask) of LB medium (SIGMA ALDRICH; St. Louis, Mo.) 20 gm/L, containing 50 mg/L Spectinomycin; 10 mg/L Rifampicin; and 50 mg/L Streptomycin with shaking (250 rpm in the dark) at 26° for 6 hr. This culture was established by 1:5 dilution of a 25 mL overnight culture (grown in the same medium) into the fresh medium. Cells were pelleted by centrifugation for 15 min at 3500 rpm at 4°, then uniformly resuspended (by gently pipetting up and down) in Inoculation Medium (1 nM) to an optical density of approximately 1.0 at 600 nm (OD₆₀₀). Inoculation Medium contains: 2.2 gm/L MS salts (Frame et al. (2011, Genetic Transformation Using Maize Immature Zygotic Embryos. In Plant Embryo Culture Methods and Protocols: Methods in Molecular Biology. T. A. Thorpe and E. C. Yeung, (Eds), Springer Science and Business Media, LLC. pp 327-341); 1×ISU Modified MS Vitamins (Frame et al., 2011 supra); 68.4 gm/L sucrose; 36 gm/L glucose; 115 mg/L L-proline; 100 mg/L myo-inositol; and 200 μM acetosyringone (prepared in DMSO); at pH 5.4. Depending upon the experiment, an appropriate amount of surfactant solution (e.g. BREAK-THRU® S 233 at 0.01% final concentration) was added to the Inoculation Medium after suspending the cells.

The Agrobacterium and embryo solution was incubated for 5 to 15 minutes at room temperature, and then the embryos were transferred to Co-cultivation Medium, which contained 4.33 gm/L MS salts; 1×ISU Modified MS Vitamins; 30 gm/L sucrose; 700 mg/L L-proline; 3.3 mg/L Dicamba in KOH (3,6-dichloro-o-anisic acid or 3,6-dichloro-2-methoxybenzoic acid); 100 mg/L myo-inositol; 100 mg/L Casein Enzymatic Hydrolysate; 15 mg/L AgNO₃; 100 μM acetosyringone in DMSO; and 3 gm/L GELZAN™ (SIGMA-ALDRICH); at pH 5.8. Co-cultivation incubation was for 3 to 4 days at 25° under continuous white fluorescent light (approximately 50 μEm⁻²s⁻¹).

Resting and Selection

After co-cultivation, the embryos (36 embryos/plate) were carefully transferred to non-selection Resting Medium containing 4.33 gm/L MS salts; 1×ISU Modified MS Vitamins; 30 gm/L sucrose; 700 mg/L L-proline; 3.3 mg/L Dicamba in KOH; 100 mg/L myo-inositol; 100 mg/L Casein Enzymatic Hydrolysate; 15 mg/L AgNO₃; 0.5 gm/L MES (2-(N-morpholino)ethanesulfonic acid monohydrate (PHYTOTECHNOLOGIES LABR.; Lenexa, Kans.); 250 mg/L Carbenicillin; and 2.3 gm/L GELZAN™; at pH 5.8. Incubation was continued for 7 days at 28° in continuous white fluorescent light conditions as above.

Following the 7 day resting period, the embryos were transferred to Selection Medium. For selection of maize tissues transformed with a superbinary plasmid containing a plant expressible aad1 selectable marker gene, the embryos (18 embryos/plate) were first transferred to Selection Medium I which consisted of the Resting Medium (above), and containing 100 nM R-Haloxyfop acid (0.0362 mg/L). The embryos were incubated for 1 week, and then transferred (12 embryos/plate) to Selection Medium II, which consisted of Resting Medium (above), and with 500 nM R-Haloxyfop acid (0.1810 mg/L), on which they were incubated for an additional 2 weeks. Transformed isolates were obtained over the course of approximately 4 to 6 weeks at 28° under 24-hour white fluorescent light conditions (approximately 50 μEm⁻²s⁻¹). Recovered isolates were transferred to fresh Pre-Regeneration medium for initiation of regeneration and further analysis.

Those skilled in the art of maize transformation will understand that other methods of selection of transformed plants are available when other plant expressible selectable marker genes (e.g. herbicide tolerance genes) are used.

Pre-Regeneration

Following the selection process, cultures exposed to the 24-hour light regime were transferred (6 to 8 calli/plate) to Pre-regeneration Medium containing 4.33 gm/L MS salts; 1×ISU Modified MS Vitamins; 45 gm/L sucrose; 350 mg/L L-proline; 100 mg/L myo-inositol; 50 mg/L Casein Enzymatic Hydrolysate; 1.0 mg/L AgNO₃; 0.25 gm/L MES; 0.5 mg/L naphthaleneacetic acid in NaOH; 2.5 mg/L abscisic acid in ethanol; 1 mg/L 6-benzylaminopurine; 250 mg/L Carbenicillin; 2.5 gm/L GELZAN™; and 500 nM R-Haloxyfop acid; at pH 5.8. Incubation was continued for 7 to 14 days at 28° continuous white fluorescent light (approximately 50 μEm⁻²s⁻¹).

Regeneration and Plantlet Isolation

For regeneration, the cultures were transferred (up to 12 calli per PHYTATRAY™ (PHYTOTECHNOLOGIES LABR.)) to a primary Regeneration Medium containing 4.33 gm/L MS salts; 1×ISU Modified MS Vitamins; 60 gm/L sucrose; 100 mg/L myo-inositol; 125 mg/L Carbenicillin; 3.5 gm/L GELLAN GUM G434 (PHYTOTECHNOLOGIES LABR.); and 500 nM R-Haloxyfop acid; at pH 5.8 and plantlets were allowed to generate and grow for up to 3 weeks.

When plantlets reached 3 to 5 cm in length, they were transferred (6 plants per PHYTATRAY™) to Plant Growth Medium containing 4.33 gm/L MS salts; 1×ISU Modified MS Vitamins; 30 gm/L sucrose; 100 mg/L myo-inositol; 3.5 gm/L GELLAN GUM G434; and 0.5 mg/L indoleacetic acid in NaOH; at pH5.8, and incubated at 25° under 16-hour white fluorescent light conditions (approximately 50 μEm⁻²s⁻¹) to allow for further growth and development of the shoot and roots.

Seed Production

Plants were transplanted into METRO-MIX 360 soilless growing medium (SUN GRO HORTICULTURE; BELLEVUE, WA) and hardened-off in a growth room. Plants were then transplanted into SUNSHINE CUSTOM BLEND 160 soil mixture and grown to flowering in the greenhouse. Controlled pollinations for seed production were conducted.

### Example 4

### Transformation Efficiencies Using Agrobacterium Cells Grown in Liquid Medium

Agrobacterium superbinary strain LBA4404(pEPS1083) was used to transform maize immature embryos by the method disclosed in Example 2 (Transformation Protocol 1). Comparisons were made of the transformation efficiencies obtained when the Agrobacterium cells were scraped from YEP agar plates and resuspended in Infection Medium (IfM), versus experiments done at the same time using Agrobacterium cells grown in liquid medium LB, harvested by centrifugation, and resuspended in IfM. Comparative transformation efficiencies were determined at various stages of the process by counting the numbers of yellow fluorescent spots (YFP+) on treated tissue pieces one to five weeks after initiation of the transformation experiments. Table 2 summarizes the results obtained.

TABLE 2
Comparison of transformation efficiencies using Agrobacterium LBA4404(pEPS1083)
inocula prepared from cells scraped from agar plates or harvested after growth in liquid culture.

| Experiment | Stage of | Agro. | No. of | No. of |
|---|---|---|---|---|
| Number | YFP Count | Growth | Embryos Treated | YFP⁺ Calli (%) |
| 1 | Selection | Plate | 108 | 40/108 (37) |
| | Medium I | Liquid | 103 | 103/103 (100) |
| | | Plate | 108 | 66/108 (61) |
| | | Liquid | 36 | 36/36 (100) |
| 2 | Pre-Regeneration | Plate | 76 | 0/76 (0) |
| | Medium | Liquid | 83 | 32/83 (39) |
| | | Plate | 107 | 0/107 (0) |
| | | Liquid | 100 | 18/100 (18) |
| 3 | Pre-Regeneration | Plate | 71 | 0/71 (0) |
| | Medium | Liquid | 72 | 26/72 (36) |
| | | Plate | 91 | 0/91 (0) |
| | | Liquid | 78 | 7/78 (9) |
| 4 | Regeneration | Plate | 73 | 4/73 (5) |
| | Medium I | Liquid | 97 | 51/97 (53) |
| | | Plate | 69 | 3/69 (4) |
| | | Liquid | 59 | 36/59 (61) |

The results summarized in Table 2 demonstrate that infection of maize embryos using Agrobacterium cells freshly harvested from liquid culture provides significantly higher transformation efficiencies than is obtained using cells scraped from agar plates.

### Example 5

### Improvement of Transformation Efficiencies by Addition of Surfactant to Transformation Protocol 1

Agrobacterium superbinary strain LBA4404(pDAB108652) was used to transform maize immature embryos by the methods disclosed in Example 2. Plasmid pDAB108652 contains the YFP coding region, whose expression was driven by the ZmUbi1 promoter, and also harbors the aad1 herbicide tolerance coding region under expression control of the rice actin1 promoter. Comparisons were made of the transformation efficiencies obtained when the Agrobacterium cells were suspended in IfM lacking surfactant, versus experiments done at the same time with IfM containing added surfactant BREAK-THRU® S 233 at various concentrations. Transformation efficiencies were calculated by counting calli with fluorescent sectors (each callus arising from a single embryo) after 4 weeks of Haloxyfop selection. At this time, the fluorescent sectors were large and therefore the tissues represented stably transformed sectors. The results summarized in Table 3 demonstrate that use of surfactant increases transformation efficiencies, and that there is a sensitivity of the enhancing effect on the concentration of surfactant used.

TABLE 3
Effect of various concentrations of surfactant BREAK-THRU ®
S 233 on transformation efficiencies.

| | | | No. | |
|---|---|---|---|---|
| Experiment | | Surfactant | Embryos | Transformation |
| Number | Plasmid | Concentration | Treated | Efficiency (%) |
| 1 | pDAB108652 | 0% | 245 | 2.86% |
| | | 0.005% | 126 | 14.29% |
| | | 0.01% | 129 | 8.53% |
| 2 | pDAB108652 | 0% | 272 | 0.74% |
| | | 0.02% | 135 | 6.67% |
| | | 0.04% | 140 | 0% |

Agrobacterium superbinary strain LBA4404(pEPS1083) was used to transform maize immature embryos by the method disclosed in Example 2. Comparisons were made of the transformation efficiencies obtained when the Agrobacterium cells were suspended in IfM lacking surfactant, versus experiments done at the same time in the presence of added surfactant in the IfM. Comparative transformation efficiencies were determined at various stages of the process by counting the numbers of yellow fluorescent spots (YFP+) on treated tissue pieces one to five weeks after initiation of the transformation experiments. Table 4 summarizes the results obtained.

In some experiments, the Agrobacterium cells were washed with IfM (with, or without, surfactant) before the cocultivation step by suspension and gentle centrifugation (“wash” in Table 4). Further, in Experiment 5 (Table 4) 200 μM acetosyringone, (rather than 100 μM as is specified in Example 2) was used to induce vir gene expression, and the Agrobacterium cells were grown on a plate of LB medium with appropriate antibiotics, rather than YEP medium.

TABLE 4
Enhancement of transformation efficiency by Agrobacterium through the use of a
surfactant. Surfactants BREAK-THRU ® S 233, PREFERENCE ® or TACTIC ™ were added to
the Infection Medium (working concentration 0.01%) used to create a suspension of
Agrobacterium cells prior to co-cultivation.

| | | | No. of | No. of | % of |
|---|---|---|---|---|---|
| | Stage of YFP | | Embryos | Embryos | Embryos |
| EXP | Count | Treatment | Treated | YFP+ | YFP+ |
| 1 | Resting | No surfactant | 50 | 28 | 56 |
| | Medium | S 233* | 48 | 33 | 69 |
| | | PREFERENCE ® | 48 | 29 | 60 |
| 2 | Selection | No surfactant | 60 | 10 | 17 |
| | Medium I | S 233 | 60 | 39 | 65 |
| 3 | Selection | No surfactant | 64 | 20 | 31 |
| | Medium I | S 233 + IfM wash** | 60 | 44 | 73 |
| | | IfM + S 233 wash | 66 | 24 | 36 |
| | | S 233 + S 233 wash | 72 | 55 | 76 |
| 4 | Selection | No surfactant | 36 | 0 | 0 |
| | Medium I | S 233 + IfM wash | 36 | 4 | 11 |
| | | IfM + S 233 wash | 36 | 8 | 22 |
| | | S 233 + S 233 wash | 36 | 19 | 53 |
| | | | No. of | No. of | % of |
| | Stage of YFP | | Embryos | Calli | Embryos |
| EXP | Count | Treatment | Treated | YFP+ | YFP+ |
| 5*** | Selection | No surfactant | 60 | 3/10 | 5 |
| | Medium II | S 233 | 60 | 29/46 | 48 |
| 6 | Selection | No surfactant | 193 | 4/118 | 3 |
| | Medium II | S 233 | 193 | 15/78 | 19 |
| 7 | Pre-Regeneration | No surfactant | 132 | 4/76 | 5 |
| | Medium | S 233 | 110 | 32/74 | 43 |
| | | TACTIC ™ | 114 | 8/60 | 13 |

*S 233 is BREAK-THRU ® S 233
**IfM is Infection Medium used to suspend and/or wash the Agrobacterium cells.
***The Agrobacterium cells were grown on an LB medium plate with antibiotics and vir gene expression was induced with 200 μM acetosyringone.

The experiments summarized in Table 4 clearly show that the presence of surfactant BREAK-THRU® S 233 in the Infection Medium used to re-suspend the Agrobacterium cells scraped from solid medium plates dramatically increases the transformation efficiencies of immature embryos. Further, surfactant TACTIC™ has a positive but less dramatic effect on enhancing transformation efficiency.

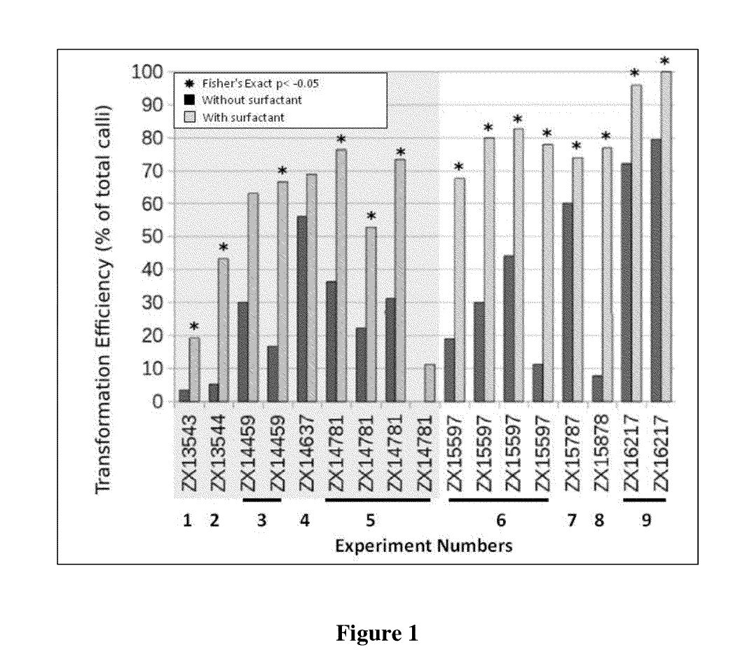

In a further exemplification of the methods of this disclosure, immature maize embryos were transformed with cells of Agrobacterium strain LBA4404(pEPS1083) by the methods of Example 2. Transformation efficiencies were monitored by the appearance of YFP+ spots or sectors on developing calli from immature embryos. The left side of FIG. 1 shows five experiments (Experiments 1 to 5) using Agrobacterium cells scraped from solid agar plates, and the right side of FIG. 1 shows results from three experiments (Experiments 6 to 9) in which the Agrobacterium cells were harvested from liquid-grown cultures. In combined Experiments 1 through 5, transformation efficiencies were increased in embryos from all nine of the ears harvested (100%) and the transformation efficiency increases were statistically significant (Fisher's Exact p<−0.05) in embryos from six of the nine ears (67%). In combined Experiments 6 through 9 (liquid grown Agrobacteria), embryos from all eight of the ears harvested (100%) showed a statistically significant increase in transformation efficiency. Thus, it is clear from the results summarized in FIG. 1 that addition of BREAK-THRU® S 233 to the Infection Medium dramatically increases transformation efficiencies of maize immature embryos, in some cases resulting in transformation efficiencies of over 90%.

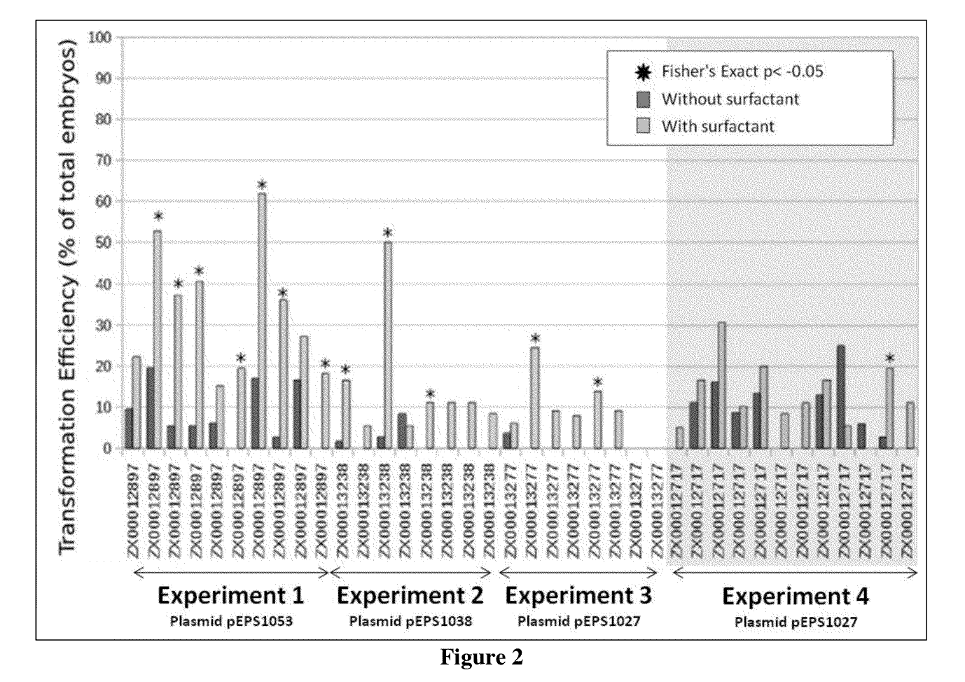

In another illustration of the methods of this disclosure, immature maize embryos were transformed with cells of Agrobacterium strain LBA4404 harboring various plasmids (all of which contained the aad1 selectable marker gene) by the methods of Example 2. As before, the experimental treatments compared transformation efficiencies with, or without, the use of 0.01% surfactant BREAK-THRU® S 233. The embryos were regenerated and taken all the way through Haloxyfop selection to plant production. Thus, the data were collected at a substantially later stage than that summarized in FIG. 1. Transformation efficiency percentages were calculated by dividing the number of embryos that produced a transgenic plant (“an event”) by the number of treated immature embryos, times 100. For this purpose, an embryo was counted as a single event even if it produced multiple transgenic plants. The results of three experiments using Agrobacterium cells scraped from agar plates are shown in FIG. 2 (Experiments 1, 2 and 3). In addition, FIG. 2 shows the results of an experiment (Experiment 4) in which the Agrobacterium cells were grown in liquid medium, harvested by centrifugation, and resuspended in IM (with, or without, BREAK-THRU® S 233). Paired bars in FIG. 2 show the responses of embryos from individual ears.

It is clear from the data in FIG. 2 that addition of the surfactant BREAK-THRU® S 233 results in a dramatic increase in Agrobacterium-mediated transformation efficiencies of maize immature embryos, regardless of previous growth configuration of the Agrobacterium cells and regardless of the gene composition of the transforming plasmid. In combined Experiments 1, 2, and 3, transformation efficiencies were increased in embryos from 23 of the 26 ears harvested (88%) and the transformation efficiency increases were statistically significant (Fisher's Exact p<−0.05) in embryos from 12 of the 26 ears (46%). In Experiment 4 (liquid growth Agrobacteria) embryos from 10 of the 12 ears harvested (83%) showed an increase in transformation efficiency, and the increase was statistically significant in one of the 12 ears (8%).

### Example 6

### Comparison of Transformation-Enhancing Action of Surfactants of Various Chemical Classes

Table 1 provides a non-limiting list of surfactants of several chemical classes. Transformation experiments of immature embryos were conducted using Transformation Protocol 2 as provided in Example 3. Agrobacterium cells harboring various plasmids were suspended in Inoculation Medium (1 nM) containing BREAK-THRU® S 233 or various other surfactants (all at a concentration of 0.01%), and transformation rates (measured by a Taqman® assay of the aad1 gene were compared 7 to 10 weeks after initiation of the experiment. Transformation efficiency percentages were calculated by dividing the number of embryos that produced a transgenic plant (“an event”) by the number of treated immature embryos, times 100. For this purpose, an embryo was counted as a single event even if it produced multiple transgenic plants. Table 5 presents the transformation efficiencies obtained.

TABLE 5
Comparison of transformation efficiencies obtained with surfactants
of various chemical classes. BREAK-THRU ® S 233 and other
surfactants were used at a concentration of 0.01%.

| | | | No. of | Transformation |
|---|---|---|---|---|
| Experiment | | | Embryos | Efficiency |
| Number* | Plasmid | Surfactant | Treated | (%) |
| 1 | pEPS1036 | BREAK-THRU ® S 233 | 756 | 25 |
| | | SILWET ® HS429 | 498 | 4.1 |
| 2 | pEPS6008 | BREAK-THRU ® S 233 | 648 | 16.4 |
| | | AGNIQUE ® SBO-10 | 648 | 14.5 |
| 3 | pEPS1066 | BREAK-THRU ® S 233 | 648 | 15 |
| | | SILWET ® HS429 | 648 | 5 |
| 4 | pEPS1068 | BREAK-THRU ® S 233 | 647 | 14.4 |
| | | ADSEE ® AB-650 | 660 | 1.9 |
| 5 | pEPS6004 | BREAK-THRU ® S 233 | 534 | 23 |
| | | TRYCOL ® 5941 | 540 | 15 |
| 6 | pEPS1013 | BREAK-THRU ® S 233 | 648 | 20 |
| | | METASPERSE ® 500 L | 648 | 9 |
| 7 | pEPS1064 | BREAK-THRU ® S 233 | 390 | 16.3 |
| | | UPTAKE ® | 324 | 5.6 |
| 8 | pEPS1018 | BREAK-THRU ® S 233 | 432 | 9 |
| | | ALCOSPERSE ® 725 | 432 | 0 |
| 9 | pEPS1038 | BREAK-THRU ® S 233 | 427 | 23 |
| | | GERENOL ® CF/AS 30 | 432 | 27 |
| 10 | pEPS1059 | BREAK-THRU ® S 233 | 468 | 29 |
| | | SILWET ® 618 | 324 | 14 |
| 11 | pEPS1028 | BREAK-THRU ® S 233 | 408 | 34.3 |
| | | UPTAKE ® | 408 | 25.7 |
| 12 | pEPS1064 | BREAK-THRU ® S 233 | 428 | 30.1 |
| | | SILWET ® 625 | 432 | 14.4 |

*On average 4 ears were used in a given experiment. Embryos harvested from a single ear were split between the two treatments in each experiment.

The results summarized in Table 5 demonstrate that the use of BREAK-THRU® S 233, when included in the Inoculation Medium used to re-suspend the Agrobacterium inoculum cells grown in and harvested from liquid medium, provided transformation efficiencies that were superior to those obtained with the majority of the other surfactants tested. In three experiments (Experiment 2, Experiment 9, and Experiment 11), the transformation efficiencies observed were nearly the same between the two surfactants.

### Example 7

### Transformation Results from Different Operators

One skilled in the art of maize transformation will understand that plant transformation methodologies often require considerable expertise that is acquired over months or years of experimentation. Transformation efficiencies may vary over a wide range due to inconsistencies in the ways in which procedures are practiced by different operators. Thus, it is advantageous to provide maize transformation procedures that improve predictability in transformation efficiencies obtained by different operators at different times. Transformations of maize immature embryos were performed over a period of several months using the methods of Example 3 (Agrobacterium strain LBA4404 harboring various plasmids) and containing BREAK-THRU® S 233 in the Inoculation Medium. Transformation efficiencies were estimated from counts of Haloxyfop-tolerant callus tissues obtained. Table 6 summarizes the results obtained.

TABLE 6
Transformation efficiencies obtained by multiple operators using methods
that incorporate surfactant in the Inoculation Medium.

| | No. of | | | Estimated | |
|---|---|---|---|---|---|
| Operator | Ears | No. of | Total | Trans Eff. | Standard |
| ID | Used | Embryos | Events | (%)* | Deviation |
| 1 | 52 | 6466 | 2172 | 33.6 | 0.21 |
| 2 | 118 | 13069 | 5009 | 38.3 | 0.20 |
| 3 | 135 | 14525 | 5519 | 38.0 | 0.19 |
| 4 | 146 | 16874 | 6253 | 37.1 | 0.22 |
| 5 | 152 | 16463 | 5671 | 34.4 | 0.18 |
| 6 | 1 | 72 | 27 | 37.5 | 0.00 |
| 7 | 20 | 1840 | 716 | 38.9 | 0.14 |
| 8 | 22 | 2516 | 645 | 25.6 | 0.15 |
| 9 | 48 | 3779 | 1143 | 30.2 | 0.20 |
| 10 | 76 | 72 | 33 | 45.8 | 0.18 |
| Totals | 770 | 75676 | 27188 | 36.0** | |

*From manual event counts—not all events were verified by Taqman ® analysis. Escape rate for Haloxyfop selection is approximately 5%.
**Average Transformation Efficiency for all operators.

The results summarized in Table 6. show that the transformation protocol disclosed in Example 3, when practiced with the inclusion of BREAK-THRU® S 233 in the Inoculation Medium, provides a robust and predictable methodology that reduces operator-to operator variation in transformation efficiency. Further, the improved predictability of the methods allows a more accurate determination of the size of the experiment (e.g. numbers of embryos that must be treated) to obtain a desired outcome (e.g. numbers of transformed events obtained).

The present invention is not limited in scope by the embodiments disclosed herein which are intended as illustrations of a few aspects of the invention and any embodiments which are functionally equivalent are within the scope of this invention. Various modifications of the methods in addition to those shown and described herein will become apparent to those skilled in the art and are intended to fall within the scope of the appended claims. Further, while only certain representative combinations of the method steps disclosed herein are specifically discussed in the embodiments above, other combinations of the method steps will become apparent to those skilled in the art and also are intended to fall within the scope of the appended claims. Thus a combination of steps may be explicitly mentioned herein; however, other combinations of steps are included, even though not explicitly stated. Where a value is explicitly recited, it is to be understood that values which are about the same quantity or amount as the recited value are also within the scope of the invention. Where a range of values is recited, each intervening integer value, and each fraction thereof, between the recited upper and lower limits of that range is also specifically disclosed, along with each sub-range between such values. The term “comprising” and variations thereof as used herein is used synonymously with the term “including” and variations thereof and are open, non-limiting terms. As used herein, the terms “modify” or “alter”, or any forms thereof, mean to modify, alter, replace, delete, substitute, remove, vary, or transform.

What is claimed is:

1. A method for plant cell transformation comprising exposing plant cells to Agrobacterium cells in a liquid medium containing a surfactant, the surfactant having a concentration of 0.001 weight percent to 0.08 weight percent in the liquid medium.

2. The method for plant cell transformation of claim 1, further comprising an additional surfactant.

3. The method for plant cell transformation of claim 1, wherein the surfactant is an adjuvant, a non-ionic surfactant, an anionic surfactant, an oil based surfactant, an amphoteric surfactant, or a polymeric surfactant.

4. The method for plant cell transformation of claim 1, wherein the surfactant is a non-ionic trisiloxane surfactant.

5. The method for plant cell transformation of claim 1, wherein the surfactant is a trisiloxane alkoxylate, ethoxylated soybean oil, alcohol ethoxylate C-13, C₁₂-C₁₄-alkyldimethyl betaines, or di-sec-butylphenol ethylene oxide-propylene oxide block co-polymer.

6. The method for plant cell transformation of claim 1, wherein the plant cells are maize cells.

7. The method for plant cell transformation of claim 1, wherein the plant cells are derived from immature embryos.

8. The method for plant cell transformation of claim 7, wherein the immature embryos are greater than or equal to 1.5 mm and less than or equal to 2.5 mm in length.

9. The method for plant cell transformation of claim 1, wherein the plant cells are exposed to continuous light after exposure to the Agrobacterium cells.

10. A method for plant cell transformation comprising:
preparing a liquid medium containing a surfactant, the surfactant having a concentration of 0.001 weight percent to 0.08 weight percent in the liquid medium;
suspending Agrobacterium cells in the liquid medium; and
exposing plant cells to the Agrobacterium cells in the liquid medium containing the surfactant.

11. The method for plant cell transformation of claim 10, wherein the Agrobacterium cells are scraped from a solid medium prior to being suspended in the liquid medium containing a surfactant.

12. The method for plant cell transformation of claim 10, wherein the Agrobacterium cells are grown in a liquid growth medium prior to being suspended in the liquid medium containing a surfactant.

13. The method for plant cell transformation of claim 10, further comprising an additional surfactant.

14. The method for plant cell transformation of claim 10, wherein the surfactant is an adjuvant, a non-ionic surfactant, an anionic surfactant, an oil based surfactant, an amphoteric surfactant, or a polymeric surfactant.

15. The method for plant cell transformation of claim 10, wherein the surfactant is a non-ionic trisiloxane surfactant.

16. The method for plant cell transformation of claim 10, wherein the surfactant is a trisiloxane alkoxylate, ethoxylated soybean oil, alcohol ethoxylate C-13, C₁₂-C₁₄-alkyldimethyl betaines, or di-sec-butylphenol ethylene oxide-propylene oxide block co-polymer.

17. The method for plant cell transformation of claim 10, wherein the plant cells are maize cells.

18. The method for plant cell transformation of claim 10, wherein the plant cells are derived from immature embryos.

19. The method for plant cell transformation of claim 18, wherein the immature embryos are 1.5 to 2.5 mm in length.

20. The method for plant cell transformation of claim 10, wherein the plant cells are exposed to continuous light after exposure to the Agrobacterium cells.
